(19) United States
(12) Patent Application Publication
(10) Pub. No.: US 2018/0051309 A1
(43) Pub. Date: Feb. 22, 2018

(54) METHODS TO ASSESS MONITOR AND CONTROL BACTERIAL BIOFILMS

(71) Applicant: ExxonMobil Research and Engineering Company, Annandale, NJ (US)

(72) Inventors: Giovanni PILLONI, Jersey City, NJ (US); Mohor CHATTERJEE, Annandale, NJ (US); Kuang HE, Ann Arbor, MI (US)

(21) Appl. No.: 15/671,383

(22) Filed: Aug. 8, 2017

Related U.S. Application Data
(60) Provisional application No. 62/376,504, filed on Aug. 18, 2016.

(51) Int. Cl. C12Q 1/02; C12Q 1/68; C09K 8/524

(52) U.S. Cl. CPC C12Q 1/02; C12Q 1/689; C12Q 2600/158; C09K 8/524; C12Q 1/6837

(57) ABSTRACT

The present disclosure describes methods for detecting, monitoring, and modulating (e.g., promoting, enhancing, sustaining, maintaining, reducing, mitigating or eliminating of) biofilm formation, as well as mitigating or eliminating Microbial Influenced Corrosion (MIC) of a metal surface. The method of monitoring includes: providing a test sample; detecting at least one of a prophage gene, a prophage gene expression, a prophage gene transcript, a prophage protein, a prophage metabolite, a prophage intermediate, or a combination thereof, wherein the existence, amount or level of prophage gene expression, a prophage gene transcript, a prophage protein, a prophage metabolite, a prophage intermediate, or a combination thereof relative to a control is indicative of biofilm presence, formation or the state of the biofilm in a sample. The present disclosure also describes a composition for modulating biofilm formation, maintenance, mitigation, reduction, and elimination.

### CROSS-REFERENCE TO RELATED APPLICATIONS

This application claims the benefit of U.S. Provisional Application No. 62/376,504, filed on Aug. 18, 2016, the entire contents of which are incorporated herein by reference.

### FIELD

The disclosure generally relates to methods of detecting and modulating (e.g., formation, induction, mitigation, reduction, or elimination of) biofilms on surfaces, such as the equipment used by the petroleum and natural gas industries to acquire, produce, store, transport, and process raw materials such as oil and gas. More specifically, the specification relates to methods and compositions for preventing and/or mitigating the formation of harmful biofilms associated with microbial influenced corrosion on susceptible metal surfaces of oil and gas production, storage, and transport equipment by contacting said equipment with compositions of the present disclosure or the promotion or formation of biofilms on the porous surfaces of oil reservoirs thereby, enhancing oil production.

### BACKGROUND

Bacteriophages are bacterial viruses. Some bacteriophages have two phases: a lytic phase and a lysogenic phase. During the lysogenic phase (i.e., cycle or state) the phage introduces the genetic material of the phage to the inside of the microbe. This genetic material is integrated within the host genome as a “prophage” for an indefinite amount of time and until the conditions become favorable for the bacteriophage to switch to a lytic cycle. The genetic material is passively replicated, along with the host chromosomal DNA, by the host replication enzymes and the host microbe is referred to as the lysogen.

During the lytic phase (i.e., cycle or infection), the phage-introduced genetic material inside of the microbe takes control of the biological machinery of the microbe in order to replicate itself (i.e., make copies of the infecting bacteriophage), and finally, lyse and kill the microbe, thereby releasing more viruses (i.e., bacteriophages), which can restart the cycle.

Biofilm formation is a bacterial adaptation that facilitates colonization of living and non-living surfaces. Bacterial cells are embedded within a self-produced matrix of extracellular polymeric substance (EPS), including extracellular DNA, proteins and polysaccharides that bind the cells together and to a surface. The biofilm matrix can also protect the bacteria from, e.g., antibiotics. Biofilm formation may be initiated in response to many factors, such as cellular recognition of a specific or non-specific attachment site on a surface, nutritional cues, exposure to sub-inhibitory concentrations of antibiotics, etc. In certain circumstances, biofilms can be considered beneficial. For example, biofilms can be utilized to selectively clog reservoir pores, thereby facilitating microbial enhanced oil recovery (MEOR), or as a potential surface-associated biocatalyst.

However, in the refining industry, biofilms can have deleterious impacts, including being responsible for corrosion (e.g., microbial influenced corrosion (MIC)) and reservoir souring. MIC poses severe operational, environmental, and safety problems to the petroleum and/or natural gas industries, particularly with respect to corrosion of equipment used in the storage, processing, and/or transport of oil and gas crude and/or processed materials. Costs resulting from MIC in these industries due to repair and replacement of damaged equipment, spoiled oil, environmental clean-up, and injury-related health care, amount to well over several billion dollars (USD) per year.

The mechanisms by which microbial influenced corrosion causes damage are poorly understood despite many decades of research. See Kwan Li et al., “Beating the bugs: Roles of microbial biofilms in corrosion,” Corrosion Reviews, Vol. 31, Issue 3-6, December 2013, pp. 73-84 (the contents of which are incorporated by reference). However, it is believed that microbial influenced corrosion is primarily caused by the formation of microbial biofilms on equipment metal surfaces that come into contact with crude oil and gas and/or the liquid systems involved in refining crude oil and gas.

The microorganisms thought to be primarily responsible for corrosion at least in an anaerobic environment within the oil industry are sulfate-reducing bacteria. Other culpable bacteria include iron oxidizing bacteria, sulfur oxidizing bacteria, nitrate reducing bacteria, methanogens, and acid producing bacteria, among others. These categories of bacteria generally are capable of reducing metal directly producing metabolic products that are corrosive (e.g., hydrogen sulfide gas), and/or leading to the formation of biofilms that indirectly alter the local environment to promote corrosion. See N. Muthukumar et al., “Microbiologically influenced corrosion in petroleum product pipelines—A Review,” Indian Journal of Experimental Biology, Vol. 41, September 2003, pp. 1012-1022.

Sulfate-reducing bacteria, in particular, are ubiquitous and can grow in almost any environment. They are routinely found in waters associated with oil production systems and can be found in virtually all industrial aqueous processes, including cooling water systems and petroleum refining. Sulfate-reducing bacteria require an anaerobic (oxygen-free) aqueous solution containing adequate nutrients, an electron donor, and electron acceptor. A typical electron acceptor is sulfate, which produces hydrogen sulfide upon reduction. Hydrogen sulfide is a highly corrosive gas and reacts with metal surfaces to form insoluble iron sulfide corrosion products. In addition, hydrogen sulfide partitions into the water, oil, and natural gas phases of produced fluids and creates a number of serious problems. For instance, “sour” oil and gas (i.e., reservoir souring), which contains high levels of hydrogen sulfide, have a lower commercial value than low sulfide oil and gas. Removing biogenic hydrogen sulfide from sour oil and gas increases the cost of these products. It is also an extremely toxic gas and is immediately lethal to humans at even small concentrations. Thus, its presence in the oil field poses a threat to worker safety.

Corrosion—often characterized in association with pitting of metal surfaces—caused by sulfate-reducing bacteria frequently results in extensive damage to oil and gas storage, production, and transportation equipment. Pipe systems, tank bottoms, and other pieces of oil production equipment can rapidly fail if there are areas where microbial corrosion is occurring. If a failure occurs in a pipeline or oil storage tank bottom, the released oil can have serious environmental consequences. Also, if a failure occurs in a high pressure water or gas line, the consequences can be worker injury or death. Any failure at least involves repair or replacement costs.

A variety of strategies have been developed to mitigate the corrosive effects of MIC and/or the biofilms that contribute or cause MIC. Such techniques include the use of corrosion-resistant metals, temperature control, pH control, radiation, filtration, protective coatings with corrosion inhibitors or other chemical controls (e.g., biocides, oxidizers, acids, alkalis), bacteriological controls (e.g., phages, enzymes, parasitic bacteria, antibodies, competitive microflora), pigging (i.e., mechanical delamination of corrosion products), anodic and cathodic protection, and modulation of nutrient levels. However, each of these existing methods face obstacles, such as, high cost, lack of effectiveness, short life-span, or requirement for repeat applications. For example, regular biocide injections are only effective sometimes and only in particular environments. In addition, biocides often fail due to incompatibility with other commonly used corrosion inhibitors and because of biofilm permeability issues, i.e., the biocides are unable to penetrate or permeate the biofilms due to the properties of the extracellular matrix of the biofilm. Also, many of the above controls are not practical for implementing in the oil field due to the potential effect on the downstream processes.

Pigging and biocide are the most commonly used approaches for controlling biofilm and corrosion in the oil field. Pigging is required to remove or disrupt the biofilm on the pipe surfaces. Pigging can also remove many of the harmful iron sulfide deposits. While pigging will be substantially effective where thick biofilms are present, thin biofilms and thin iron sulfide deposits are not appreciably affected by the scraping action of pigs. Subsequently, biocides and surfactant-biocide treatments are used extensively to control bacterial activity in oil field systems. However, biocides are not typically effective in penetrating the biofilms, and therefore, have reduced effectiveness against the underlying bacteria. Combination treatments in conjunction with pigging are more effective than chemical treatments alone. However, treatments must be made routinely on a fixed schedule or else the bacteria population increases significantly and control becomes even more difficult.

MEOR is a multidisciplinary field that is aimed at manipulating the function and/or structure of microbial environments in oil reservoirs to improve the recovery of oil trapped in porous media. MEOR is a tertiary oil extraction technology that increases the life of mature oil reservoirs by allowing the crude oil recovery from depleted oil reservoirs to solve. Indigenous reservoir microbes are simulated or specially selected consortia of natural bacteria are injected into the reservoir to produce specific metabolic events that lead to improved oil recovery. Improvement in oil recovery by reservoir re-pressurization and the role of biopolymers for selective plugging of oil-depleted zones and for biofilm formation have been described.

Thus, there exists a need in the art for an improved approach for detecting biofilms, monitoring biofilms, and modulating/controlling biofilms, and in particular, methods that effectively detect, monitor, and modulate (e.g., enhance, form, reduce, mitigate, or otherwise eliminate) biofilms (e.g., pore-plugging biofilms or corrosion-associated biofilms) in the field of oil and gas production and refining, as well as in many other areas.

### SUMMARY

The specification relates, in part, to the surprising discovery that the formation or presence of a biofilm can be determined through prophage gene or protein expression/status/presence and/or prophage repressor gene expression/presence/status, and that biofilms formation and maintenance can be detected or monitored by examining prophage gene or protein expression/status/presence. Furthermore, it was surprising and unexpected that bacteria could be modulated to form, sustain (e.g., maintain), or inhibit (e.g., reduce, mitigate, or eliminate) a biofilm.

In one aspect, the disclosure relates to a method of detecting biofilm formation or monitoring the state of a biofilm. The method comprises: providing a test sample (e.g., a produced water sample); detecting at least one of a prophage gene, a prophage gene expression, a prophage gene transcript, a prophage protein, a prophage metabolite, a prophage intermediate, or a combination thereof, wherein the existence, amount or level of prophage gene expression, a prophage gene transcript, a prophage protein, a prophage metabolite, a prophage intermediate, or a combination thereof relative to a control is indicative of biofilm presence, formation or the state of the biofilm in a sample.

In some embodiments, an increase relative to levels detected in a previously taken sample is indicative of biofilm formation or an increase in biofilm burden/biomass.

In other embodiments, a decrease relative to levels detected in a previously taken sample is indicative of a decrease in biofilm burden/biomass.

In further embodiments, an increase relative to levels observed in planktonic bacteria is indicative of biofilm formation.

In certain embodiments, a lack of detection is indicative of a lack of a biofilm.

In some particular embodiments, the step of detecting comprises binding at least one of: (i) at least one labelled nucleic acid probe that hybridizes to the prophage gene, the prophage transcript, or a combination thereof (ii) at least one binding polypeptide that binds the prophage protein, the prophage metabolite, the prophage intermediate, or a combination thereof or (iii) a combination thereof. In another embodiment, (ii) further comprises: contacting the sample with the binding polypeptide, and contacting the sample with a labelled secondary polypeptide that binds specifically to the binding polypeptide. In particular embodiments, the binding polypeptide includes an antibody or an antigen-binding fragment thereof.

In other embodiments, the label is a fluorophore, an enzyme, a radioisotope, or a chemiluminescent compound.

In some embodiments, the biofilm is formed by an anaerobic bacteria.

In yet another embodiment, the anaerobic bacteria is selected from the group consisting of sulfate reducing bacteria, iron oxidizing bacteria, sulfur oxidizing bacteria, nitrate reducing bacteria, methanogens, and acid producing bacteria.

In further embodiments, the sulfate reducing bacteria is of the genera Desulfovibrio, Desulfotomaculum, Desulfosporomusa, Desulfosporosinus, Desulfobacter, Desulfobacterium, Desulfobacula, Desulfobotulus, Desulfocella, Desulfococcus, Desulfofaba, Desulfofrigus, Desulfonema, Desulfosarcina, Desulfospira, Desulfotalea, Desulfotignum, Desulfobulbus, Desulfocapsa, Desulfofustis, Desulforhopalis, Desulfoarculus, Desulfobacca, Desulfomonile, Desulfotigmum, Desulfohalobium, Desulfomonas, Desulfonatronovibrio, Desulfomicrobium, Desulfonatronum, Desulfacinum, Desulforhabdus, Syntrophobacter, Syntrophothermus, Thermaerobacter, and Thermodesulforhabdus.

In certain embodiments, the sulfate reducing bacteria is of the genera Desulfovibrio.

In another aspect, the disclosure relates to a method of modulating bacteria. The method comprises contacting the bacteria with an effective amount of a composition comprising at least one agent that: (a) activates or promotes at least one of a prophage gene, prophage gene transcription, a prophage gene expression, a prophage transcript, a prophage protein, a prophage metabolite, a prophage intermediate or a combination thereof; or (b) inhibits or inactivates at least one of a prophage gene, prophage gene transcription, a prophage gene expression, a prophage transcript, a prophage protein, a prophage metabolite, a prophage intermediate or a combination thereof; or (c) activates or promotes at least one of a prophage repressor gene, prophage repressor gene transcription, a prophage repressor transcript, a prophage repressor protein or a combination thereof; (d) inhibits or inactivates at least one of a prophage repressor gene, prophage repressor gene transcription, a prophage repressor transcript, a prophage repressor protein or a combination thereof; or (e) a combination thereof.

In some embodiments, the method further comprises detecting biofilm formation or monitoring the state of a biofilm in accordance with the present disclosure.

In other embodiments, (a) activating or promoting of the prophage gene, prophage gene transcription, the prophage gene expression, the prophage transcript, the prophage protein, the prophage metabolite, the prophage intermediate or a combination thereof results in the activation or induction of a prophage lytic cycle or inhibition of a prophage lysogenic cycle in the bacteria.

In yet further embodiments, (b) inhibiting or inactivating a prophage gene, prophage gene transcription, a prophage gene expression, a prophage transcript, a prophage protein, a prophage metabolite, a prophage intermediate or a combination thereof results in the inhibition of a prophage lytic cycle or promotion of a prophage lysogenic phase in the bacteria.

In certain embodiments, (c) activating or promoting a prophage repressor gene, prophage repressor gene transcription, a prophage repressor gene transcript, a prophage repressor protein or a combination thereof results in the inhibition of a prophage lytic cycle or promotion of a prophage lysogenic phase in the bacteria.

In particular embodiments, (d) inhibiting or inactivating a prophage repressor gene, prophage repressor gene transcription, a prophage repressor gene transcript, a prophage repressor protein or a combination thereof results in the activation or induction of a prophage lytic cycle or inhibition of a prophage lysogenic cycle in the bacteria.

In some embodiments, the activation or induction of the prophage lytic cycle or the inhibition of a prophage lysogenic cycle results in a reduction in biofilm formation or burden.

In other embodiments, inhibiting of the prophage lytic cycle or promoting the prophage lysogenic phase results in an increase in biofilm formation or burden.

In further embodiments, the agent or agents induce the prophage lytic cycle or inhibit biofilm production/maintenance or promotes biofilm degradation.

In yet another embodiment, the method further comprises a secondary treatment for reducing the formation or activity of a biofilm on a surface.

In an embodiment, the secondary treatment is selected from the group consisting of pigging, radiation treatment, pH adjustment, nutrient adjustment, and installation of corrosion-resistant metals.

In a further aspect, the disclosure relates to a composition for modulating bacteria. The composition comprises an effective amount of an agent which: (i) inhibits the transcription of at least one prophage gene or promotes the transcription of at least one prophage repressor gene; or (ii) inhibits the translation of at least one prophage gene transcript or promotes the translation of at least one prophage repressor gene transcript; or (iii) leads to the degradation of at least one prophage protein or inhibits the activity of at least one prophage protein or stabilizes of at least one prophage repressor protein or enhances the activity of at least one prophage repressor protein; or (iv) leads to the degradation of at least one prophage metabolite or inhibits the activity of at least one prophage metabolite or stabilizes at least one prophage repressor metabolite or enhances the activity of at least one prophage repressor metabolite; or (v) leads to the degradation of at least one intermediate or inhibits the activity of at least one prophage intermediate or stabilizes at least one prophage repressor intermediate or enhances the activity of at least one prophage repressor intermediate.

In some embodiments, the agent is an antibody or a binding fragment thereof.

In other embodiments, the agent is a nucleic acid that binds to at least one of the prophage gene, the prophage transcript or a combination thereof.

In certain embodiments, the composition further comprises an effective amount of at least one additional agent.

In an embodiment, the additional agent is an indole or a functionally equivalent analog or derivative thereof in an aqueous composition or a non-aqueous composition.

In further embodiments, the additional agent is an indole or a functionally equivalent analog or derivative thereof in a liquid composition with an acidic pH or a basic pH.

In particular embodiments, the additional agent is a biocide selected from the group consisting of germicides, antibiotics, antibacterials, antivirals, antifungals, antiprotozoals and antiparasites.

In a particular embodiment, the effective amount of the composition comprising indole or a functionally equivalent analog or derivative thereof provides a concentration of indole that is between about 50-500 micromolar, about 0.5-1.0 mM, about 1.0 mM-5 mM, about 2.5 mM-10 mM, about 5 mM-25 mM, about 10 mM-100 mM, or about 50 mM-1000 mM.

In yet another aspect, the disclosure relates to a method for mitigating or eliminating Microbial Influenced Corrosion of a metal surface. The method comprises contacting the metal surface with an effective amount of a liquid composition comprising at least one agent that: (i) inhibits at least one of a prophage gene expression, a prophage transcript, a prophage protein, a prophage metabolite, a prophage intermediate, or a combination thereof; (ii) inactivates a prophage gene; or (iii) a combination thereof.

In some embodiments, the Microbial Influenced Corrosion is caused by a bacterial biofilm deposited on the surface of the metal surface.

In yet a further aspect, the disclosure relates to a method for modulating biofilm formation on a surface. The method comprises contacting the reservoir surface with an effective amount of a liquid composition comprising at least one agent that: (i) inhibits at least one of a prophage gene expression, a prophage transcript, a prophage protein, a prophage metabolite, a prophage intermediate, or a combination thereof; (ii) inactivates a prophage gene; (iii) promotes prophage repressor gene transcription, a prophage repressor gene transcript, a prophage repressor protein or a combination thereof results in the inhibition of a prophage lytic cycle or promotion of a prophage lysogenic phase in the bacteria; (iv) activates a prophage repressor gene; or (v) a combination thereof. In a particular embodiment, the surface is a metal surface. In a further embodiment, the surface is a reservoir surface (e.g., a porous surface of a reservoir).

Where applicable or not specifically disclaimed, any one of the embodiments described herein are contemplated to be able to combine with any other one or more embodiments, even though the embodiments are described under different aspects of the invention. These and other embodiments are disclosed or are obvious from and encompassed by, the following Detailed Description, including the Drawings and Examples herein.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows the normalized gene expression of two prophage regions in planktonic and biofilm samples, as further discussed in Example 1.

FIG. 2 shows the phylogenetic affiliation of two prophage regions selected from Desulfovibrio vulgaris in Example 1 (DVU0202 and DVU2867, in bold) with 33 exemplary bacterial species, as further discussed in Example 2.

### DETAILED DESCRIPTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

The specification relates, in part, to the surprising discovery that the formation or presence of a biofilm can be determined through the existence, amount or level of prophage gene or protein expression. It was also surprising and unexpected that biofilm formation and maintenance can be detected or monitored by examining expression, existence, amount or level of a prophage gene(s), transcript(s), and/or protein(s). The disclosure also relates to the surprising discovery that bacteria could be modulated to form, sustain (e.g., maintain), or inhibit (e.g., reduce, mitigate, or eliminate) a biofilm through agents directed to prophage genes, transcripts or proteins, and/or prophage repressor genes, transcripts or proteins.

Microbial Influenced Corrosion (“MIC”)—a term of art—is frequently observed at oil production sites and in transport pipelines, among other types of equipment involved in the oil production industry. MIC poses severe operational, environmental, and safety problems to the petroleum and/or natural gas industries, particularly with respect to corrosion of equipment used in the storage, processing, and/or transport of oil and gas crude and/or processed materials. Costs resulting from MIC in these industries due to repair and replacement of damaged equipment, spoiled oil, environmental clean-up, and injury-related health care, amount to well over several billion USD per year. Biofilms that form on the surfaces of such metal components are thought to be the primary causative agent triggering such corrosion as many biofilm forming environmental bacteria-particularly those in anaerobic environments-produce harmful gases (e.g., hydrogen sulfide), acids (e.g., sulfuric acid), and other agents which are highly corrosive and also which poses health and safety concerns to those workers in the industry. Current mitigation techniques to reduce microbial-induced corrosion are available, but are not effective enough and/or are not practical in the industry due to high cost and other reasons. For example, the use of biocides is common, but their effectiveness is limited to due inability to permeate the corrosive biofilms.

### Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more agents at the same time) and time varied administration (administration of one or more agents at a time different from that of the administration of an additional agent or agents), as long as the agents are present in the area to be treated to some extent, preferably at effective amounts, at the same time. In certain preferred aspects, one or more of the present agents described herein, are co-administered in combination with at least one additional bioactive agent, especially including an antifungal, antibacterial, and/or biocide. In particularly preferred aspects, the co-administration of agents results in synergistic activity and/or therapy, including antiviral and/or pain relief activity.

As used herein, the term “biocide” refers to a chemical substance or microorganism which can deter, render harmless, or exert a controlling effect on any harmful organism by chemical or biological means. Biocides include those that are synthetic, but also those which are naturally obtained, e.g., obtained or derived from bacteria and plants. Biocides can include, but are not limited to, germicides, antibiotics, antibacterials, antivirals, antifungals, antiprotozoals and antiparasites. Such compounds are well-known in the art and may be obtained easily from commercial sources. Reference may be made to the biocides disclosed in the book Corrosion in the Petrochemical Industry, Ed. Linda Garverick, ASM International, 1994, the contents of which are incorporated herein by reference.

As used herein, the term “Microbial Influenced Corrosion” or “MIC” or similar terms are terms in the art and shall be understood according to the meaning ascribed in the field, i.e., corrosion to metal surfaces caused directly or indirectly through the effects of bacteria and their by-products and metabolites at metal surfaces, including especially bacteria that grow on the surface of metal in a biofilm. MIC can occur in both aerobic and anaerobic conditions and generally is thought to at least require the presence of bacteria in a biofilm. MIC is considered “biotic corrosion.” MIC is also associated with surface pitting, which leads to more rapid corrosive failure than uniform corrosion.

As used herein, the term “sulfate reducing bacteria” or “SRB,” which are considered one of the main culprits of biotic corrosion in anaerobic conditions, are a grouping of bacteria that includes at least 220 species which produce H2S, and use sulfates as the terminal electron acceptor. Most SRB are considered obligate anaerobes, meaning that the cells cannot metabolize and/or replicate in the presence of oxygen, although many species can temporarily tolerate low levels of oxygen. Furthermore, anaerobic conditions capable of supporting SRB growth can be created in overall aerobic environments, due to the microniches created within the bacterial biofilm/corrosion product layer. Although SRB are the most studied and well understood of the anaerobic corrosion inducing bacteria, MIC can occur in anaerobic conditions in the absence of SRB.

As used herein, the term “corrosion-associated biofilms” refer to biofilms that have corrosive properties which contribute to Microbial Influenced Corrosion.

As used herein, the term “pigging” refers to the well-known process of intentional mechanical delamination of corrosion products and biofilm material from the surfaces of metals.

As used herein, the term “corrosion” refers to the general deterioration of a material (e.g., metallic material) due to its reaction with the environment.

As used herein, the term “indole” refers to the naturally occurring aromatic heterocyclic organic compound consisting of a six-membered benzene ring fused to a five-membered nitrogen-containing pyrrole ring, as shown in the following structure:

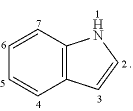

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein within the detailed description and the claims are modified by “about” or “approximately” the indicated value.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

Exemplary compositions and methods of the present invention are described in more detail below.

### Method of Detecting Biofilm Formation or Monitoring the State of a Biofilm

In one aspect, the disclosure relates to a method of detecting biofilm formation or monitoring the state of a biofilm. The method comprises: providing a test sample; detecting at least one of a prophage gene, a prophage gene expression, a prophage gene transcript, a prophage protein, a prophage metabolite, a prophage intermediate, or a combination thereof, wherein the existence, amount or level of prophage gene expression, a prophage gene transcript, a prophage protein, a prophage metabolite, a prophage intermediate, or a combination thereof relative to a control is indicative of biofilm presence, formation or the state of the biofilm in a sample. The present disclosure has demonstrated that prophage genes/proteins are overexpressed in biofilms and conversely, that prophage genes/proteins expression is downregulated in planktonic bacteria, i.e., when biofilms are treated (see the Examples below). As such, an increase relative to levels detected in a previously taken sample can be indicative of biofilm formation or an increase in biofilm burden/biomass. Conversely, a decrease relative to levels detected in a previously taken sample can be indicative of a decrease in biofilm burden/biomass.

The sample can be at least one of produced water, associated water, coupons, pig debris, surface swabs, scrapes, or a combination thereof.

Alternatively, the existence, amount or level can be compared to levels found in planktonic bacteria. For example, an increase relative to levels observed in planktonic bacteria can be indicative of biofilm formation. In certain embodiments, a lack of detection is indicative of a lack of a biofilm.

The step of detecting can comprise binding at least one of: (i) at least one labelled nucleic acid probe that hybridizes to the prophage gene, the prophage transcript, or a combination thereof; (ii) at least one binding polypeptide that binds the prophage protein, the prophage metabolite, the prophage intermediate, or a combination thereof; or (iii) a combination thereof In another embodiment, (ii) further comprises: contacting the sample with the binding polypeptide, and contacting the sample with a labelled secondary polypeptide that binds specifically to the binding polypeptide. In particular embodiments, the binding polypeptide includes an antibody or an antigen-binding fragment thereof. In another embodiments, the labeled secondary polypeptide is an antibody or an antigen-binding fragment thereof associated with a label.

The label can be one or more labeling moieties to allow for detection of hybridized probe/target polynucleotide complex or binding of the secondary polypeptide to the binding polypeptide/target protein complexe. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. In a preferred embodiment, a fluorescent dye is incorporated directly to the probe by using a fluorochrome conjugated nucleotide triphosphate (e.g. Cy3-dUTP) or through a secondary coupling reaction by first incorporating an amino allyl conjugated nucleotide triphosphate (e.g. amino allyl-dUTP) followed by chemical coupling of the fluorochrome (e.g. NHS-Cy3). In certain embodiments, the label is a fluorophore, an enzyme, a radioisotope, or a chemiluminescent compound.

The biofilm can be formed by an anaerobic bacteria, such as sulfate reducing bacteria, iron oxidizing bacteria, sulfur oxidizing bacteria, nitrate reducing bacteria, methanogens, and acid producing bacteria. The sulfate reducing bacteria can be of the genera Desulfovibrio, Desulfotomaculum, Desulfosporomusa, Desulfosporosinus, Desulfobacter, Desulfobacterium, Desulfobacula, Desulfobotulus, Desulfocella, Desulfococcus, Desulfofaba, Desulfofrigus, Desulfonema, Desulfosarcina, Desulfospira, Desulfotalea, Desulfotignum, Desulfobulbus, Desulfocapsa, Desulfofustis, Desulforhopalis, Desulfoarculus, Desulfobacca, Desulfomonile, Desulfotigmum, Desulfohalobium, Desulfomonas, Desulfonatronovibrio, Desulfomicrobium, Desulfonatronum, Desulfacinum, Desulforhabdus, Syntrophobacter, Syntrophothermus, Thermaerobacter, and Thermodesulforhabdus. In a particular embodiment, the sulfate reducing bacteria is of the genera Desulfovibrio.

### Method of Modulating Bacteria

In another aspect, the disclosure relates to a method of modulating bacteria. The method comprises contacting the bacteria with an effective amount of a composition comprising at least one agent that: (a) activates or promotes at least one of a prophage gene, prophage gene transcription, a prophage gene expression, a prophage transcript, a prophage protein, a prophage metabolite, a prophage intermediate or a combination thereof; or (b) inhibits or inactivates at least one of a prophage gene, prophage gene transcription, a prophage gene expression, a prophage transcript, a prophage protein, a prophage metabolite, a prophage intermediate or a combination thereof or (c) activates or promotes at least one of a prophage repressor gene, prophage repressor gene transcription, a prophage repressor transcript, a prophage repressor protein or a combination thereof (d) inhibits or inactivates at least one of a prophage repressor gene, prophage repressor gene transcription, a prophage repressor transcript, a prophage repressor protein or a combination thereof or (e) a combination thereof. The method of modulating bacteria can further comprise detecting biofilm formation or monitoring the state of a biofilm in accordance with the present disclosure.

Because overexpression of prophage gene(s)/protein(s) and promoting prophage protein(s) function is associated with biofilm formation, (a) activating or promoting of the prophage gene, prophage gene transcription, the prophage gene expression, the prophage transcript, the prophage protein, the prophage metabolite, the prophage intermediate or a combination thereof can result in the activation or induction of a prophage lytic cycle or inhibition of a prophage lysogenic cycle in the bacteria. Conversely, (b) inhibiting or inactivating a prophage gene, prophage gene transcription, a prophage gene expression, a prophage transcript, a prophage protein, a prophage metabolite, a prophage intermediate or a combination thereof can result in the inhibition of a prophage lytic cycle or promotion of a prophage lysogenic phase in the bacteria.

Because overexpression of prophage gene(s)/protein(s) and promoting prophage protein function(s) is associated with biofilm formation, (c) activating or promoting a prophage repressor gene, prophage repressor gene transcription, a prophage repressor gene transcript, a prophage repressor protein or a combination thereof can result in the inhibition of a prophage lytic cycle or promotion of a prophage lysogenic phase in the bacteria. Conversely, (d) inhibiting or inactivating a prophage repressor gene, prophage repressor gene transcription, a prophage repressor gene transcript, a prophage repressor protein or a combination thereof can result in the activation or induction of a prophage lytic cycle or inhibition of a prophage lysogenic cycle in the bacteria.

The activation or induction of the prophage lytic cycle or the inhibition of a prophage lysogenic cycle, as described herein, results in a reduction in biofilm formation or burden. And conversely, inhibiting of the prophage lytic cycle or promoting the prophage lysogenic phase, as described herein, results in an increase in biofilm formation or burden.

The agent or agents described herein induce the prophage lytic cycle or inhibit biofilm production/maintenance or promotes biofilm degradation as described throughout the disclosure.

The method of modulating can further comprise a step of performing a secondary treatment for reducing the formation or activity of a biofilm on a surface. For example, the secondary treatment can be an additional agent (e.g., pH adjustment, nutrient adjustment, etc.) or a mechanical treatment (e.g., pigging, etc.), a preventative treatment (e.g., installation of corrosion-resistant metals), or radiation treatment.

### Bacteria Modulating Compositions

The specification provides for compositions that may be administered to bacteria (e.g., planktonic bacteria or a biofilm on a surface) to promote or inhibit biofilm formation (e.g., inhibit or promote biofilm formation in planktonic bacteria, or inhibit or promote biofilm formation of a biofilm on a surface that needs the: (1) the effective mitigation and/or elimination of biofilms (e.g., biofilms in anaerobic conditions), (2) the effective enhancement and/or promotion of biofilms, or (3) the effective maintenance of biofilms.

Without being bound by theory, the present inventors have surprisingly discovered that promoting prophage gene/protein expression and prophage protein function enhances biofilm formation or the maintenance of an existing biofilm. In an aspect, the disclosure provides a composition for modulating bacteria. The composition comprises an effective amount of an agent which: (i) inhibits the transcription of at least one prophage gene or promotes the transcription of at least one prophage repressor gene; or (ii) inhibits the translation of at least one prophage gene transcript or promotes the translation of at least one prophage repressor gene transcript; or (iii) leads to the degradation of at least one prophage protein or inhibits the activity of at least one prophage protein or stabilizes of at least one prophage repressor protein or enhances the activity of at least one prophage repressor protein; or (iv) leads to the degradation of at least one prophage metabolite or inhibits the activity of at least one prophage metabolite or stabilizes of at least one prophage repressor metabolite or enhances the activity of at least one prophage repressor metabolite; or (v) leads to the degradation of at least one intermediate or inhibits the activity of at least one prophage intermediate or stabilizes of at least one prophage repressor intermediate or enhances the activity of at least one prophage repressor intermediate.

The agent can be an antibody or a binding fragment thereof. Alternatively, the agent can be a nucleic acid that binds to at least one of the prophage gene, the prophage transcript or a combination thereof, thereby inhibiting (e.g., siRNA, RNAi, CRISPR, etc.) or promoting gene expression.

In a further embodiment, the composition further includes an effective amount of at least one additional agent (e.g., an inhibitor of biofilms, and inhibitor of corrosion-associated biofilms, or a corrosion inhibitor). The additional agent can be a biocide selected from the group consisting of germicides, antibiotics, antibacterials, antivirals, antifungals, antiprotozoals and antiparasites. In an embodiment, the additional agent is an indole or a functionally equivalent analog or derivative thereof in an aqueous composition or a non-aqueous composition. In a certain embodiment, the additional agent is an indole or a functionally equivalent analog or derivative thereof in a liquid composition with an acidic pH or a basic pH. The composition comprising indole or a functionally equivalent analog or derivative thereof can provide a concentration of indole that is between about 50-500 micromolar, about 0.5-1.0 mM, about 1.0 mM-5 mM, about 2.5 mM-10 mM, about 5 mM-25 mM, about 10 mM-100 mM, or about 50 mM-1000 mM.

Compositions of the present disclosure can be used to reduce the formation of biofilms on surfaces, and consequently, may be used to reduce, mitigate, or eliminate Microbial Influenced Corrosion on metal surfaces, and in particular, metal surfaces on equipment involved in the storage, transport, and refinery in the petrochemical and natural gas industries. Alternatively, compositions of the present disclosure can be used to increase the formation of biofilm or sustain a biofilm on surfaces, and consequently, may be used to increase, promote or induce biofilm formation, or sustain biofilm formation, where beneficial.

The compositions of the present disclosure can modulate bacteria (e.g., reducing, eliminating, blocking, promoting, or enhancing biofilm formation in, e.g., anaerobic environments) in the process of biofilm production, which prior to the invention was not known or appreciated. The compositions of the present disclosure can be utilized to treat equipment, including pipelines, storage tanks, and refinery processing equipment. Treatments with the composition of the present disclosure can also be combined with existing microbial corrosion mitigation techniques, such as pigging and corrosion inhibitors.

Indole is widely distributed in the natural environment and can be produced by a variety of bacteria and thus, can be obtained from nature. As an intercellular signal molecule, indole regulates various aspects of bacterial physiology. The amino acid tryptophan is an indole derivative and the precursor of the neurotransmitter serotonin. Indole is a solid at room temperature. Indole can be produced by bacteria as a degradation product of the amino acid tryptophan. It occurs naturally in human feces and has an intense fecal odor. At very low concentrations, however, it has a flowery smell, and is a constituent of many flower scents (such as orange blossoms) and perfumes. It also occurs in coal tar. Indole and its derivatives can be obtained commercially from a wide range of sources that will be known the skilled artisan. Indole can also be synthesized by a variety of methods.

The disclosed methods also contemplate the use of indole analogs or equivalent compounds. As used herein, “indole equivalent” or “indole analog or functionally equivalent compound, molecule or derivative” or similar terms includes any known or yet unknown compound that has a structure that is similar to indole (or that includes one or more indole subunits) to a degree such that is produces the same or similar biological effects of indole. Such analogs or functionally equivalent compounds may be obtained in various ways, including isolation from nature, chemical modification of indole, or via chemical synthesis. The indole equivalent compound should bear at least 70%, or more preferably 75%, or even more preferably 85%, or 90%, or 95%, or 100% of the biological activity of indole. Indole analogs are well known and widely available, for example, see Timothy Barden, “Indoles: Industrial, Agricultural and Over-the-Counter Uses,” Top Heterocycl Chem (2011) 26: 31-46, which is incorporated herein by reference.

The compositions of the present disclosure for use in the disclosed methods can be prepared to have any useful properties that may be appropriate or advantageous to the particular surface to be treated, the exact composition of which will depend on various factors that include whether the surface to be treated is under aerobic or anaerobic conditions, the pH and salinity of the surface to be treated, the consortium or population characteristics of the bacteria. For, example the bacteria present in the biofilm of the target surface to be treated, the properties of the biofilm to be treated, among other characteristics.

The compositions of the present disclosure can also include other components that help stabilize and/or improve the active ingredients, or which facilitate its delivery. For example, the compositions herein described may also include surfactants or disruption agents and the like which increase the permeability and/or disruption of the biofilm to facilitate the movement of the active ingredients into the biofilm and come into contact with the bacteria therein. Surfactants are well known in the art and include anionic surfactants (e.g., ammonium lauryl sulfate, sodium lauryl sulfate (SDS, sodium dodecyl sulfate, another name for the compound), sodium lauryl ether sulfate (SLES), and sodium myreth sulfate; sodium stearate, sodium lauroyl sarcosinate), cationic surfactants (Octenidine dihydrochloride, Cetylpyridinium chloride (CPC), Benzalkonium chloride (BAC), Benzethonium chloride (BZT), 5-Bromo-5-nitro-1,3-dioxane, Dimethyldioctadecylammonium chloride, Cetrimonium bromide, Dioctadecyldimethylammonium bromide (DODAB)), and nonionic surfactants (Polyoxyethylene glycol alkyl ethers, Polyoxypropylene glycol alkyl ethers, Glucoside alkyl ethers, Polyoxyethylene glycol octylphenol ethers (e.g., Triton-X), Polyoxyethylene glycol alkylphenol ethers, Glycerol alkyl esters, Polyoxyethylene glycol sorbitan alkyl esters, Polyethoxylated tallow amine (POEA)), as well as biosurfactants (surface-active substances synthesised by living cells).

The compositions of the present disclosure are suitable for use in the disclosed methods may include any suitable amount of the active ingredients analog/equivalent. For example, compositions may be formulated in aqueous solutions having a concentration of between about 1%-5% at least one active ingredient, or between about 2.5%-10%, or between about 5%-15%, or between about 10%-25%, or between about 15%-50%, or between about 20%-75% or more. Preferably, the concentration of each active ingredient or analog/equivalent is about 5%, or preferably about 10%, or preferably about 20%, or preferably less than 50%. The skilled person will be able to determine the necessary and/or desired concentration.

When administering the composition of the present disclosure to a site targeted for treatment (i.e., a surface having MIC or MEOR), the composition is administered or delivered in an amount or dosage sufficient to provide an effective amount of the active ingredient or active ingredients. As used herein, the term “effective amount of the active ingredient or active ingredients” is the minimal amount, level, or concentration of the active ingredient or ingredients which results in a measurable or detectable effect on planktonic bacteria, biofilm bacteria, or MIC. The effective amount can be measured in terms of concentration as parts-per-million (ppm). In certain embodiments, the effective amount will be at least 0.05 ppm, or at least 0.5 ppm, or at least 1 ppm, or at least 2 ppm, or at least 4 ppm, or at least 10 ppm, or at least 15 ppm, or at least 20 ppm, or at least 20 ppm, or at least 25 ppm, or at least 50 ppm, or at least 100 ppm, or at least 1000 ppm or more.

In yet another aspect, the disclosure relates to a method for mitigating or eliminating Microbial Influenced Corrosion of a metal surface. The method comprises contacting the metal surface with an effective amount of a liquid composition comprising at least one agent that: (i) inhibits at least one of a prophage gene expression, a prophage transcript, a prophage protein, a prophage metabolite, a prophage intermediate, or a combination thereof; (ii) inactivates a prophage gene; or (iii) a combination thereof. The Microbial Influenced Corrosion can be caused by a bacterial biofilm deposited on the surface of the metal surface.

In yet another aspect, the disclosure relates to a method for modulating biofilm formation on a surface. The method comprises contacting the reservoir surface with an effective amount of a liquid composition comprising at least one agent that: (i) inhibits at least one of a prophage gene expression, a prophage transcript, a prophage protein, a prophage metabolite, a prophage intermediate, or a combination thereof (ii) inactivates a prophage gene; (iii) promotes prophage repressor gene transcription, a prophage repressor gene transcript, a prophage repressor protein or a combination thereof results in the inhibition of a prophage lytic cycle or promotion of a prophage lysogenic phase in the bacteria; (iv) activates a prophage repressor gene; or (v) a combination thereof. The surface can be a surface found in a reservoir (e.g., a porous surface of a reservoir) and/or a metal surface.

The methods disclosed herein may also include additional upstream and/or downstream testing steps that facilitate knowing whether and how to administer the composition of the present disclosure. Such additional steps may aim to determine whether a target system has a legitimate MIC risk at a particular site (e.g., crude pipeline that transports crude oil from an offshore rig to a distant domestic refinery). Other steps may also involve subsequent monitoring steps to evaluate the extent of the MIC associated biofilm, and followed then by steps to carry out a particular treatment plan with the composition of the present disclosure, e.g., an aggressive treatment plan or a lower-strength treatment plan.

For example, biofilm formation and/or corrosive damage to a pipeline might be detected as a result of regularly scheduled maintenance along a certain ten-mile stretch of crude oil pipeline. In order to learn more about the extent and nature of the biofilm burden/mass and/or damage, and therefore, an appropriate treatment, a user might sample the environmental conditions at various points along the pipeline by assessing properties that would be indicative of conditions suitable for biofilm formation, such as, (a) detection of certain bacterial species known to have a role in bacterial corrosion (e.g., sulfate reducing bacteria), (b) detection of certain corrosive metabolites (e.g., presence of organic acids, hydrogen sulfide gas, (c) existence of suitable pH and temperature conditions known to be supportive of biofilm development, (d) presence of an aqueous environment (e.g., extent of water drop-out or separation of a water phase from the crude oil), (e) slow flow rate (slower flow rates are conducive to biofilm formation), and (f) existence of high bacterial biomass. The skilled person may also wish to examine physical samples collected from the pipeline wall to detect and characterize the biofilm (e.g., thickness) or metal coupon samples placed into the flow path. Such factors can be evaluated and then assessed by the skilled person to design a specifically tailored treatment with the compositions of the present disclosure.

In some embodiments, variables affecting the specific nature of any given composition treatment can include, for example: (a) the particular agents used in the composition, (b) the concentration of the particular components of the composition (e.g., 1%, 2%, 5%, 10%, 50%, w/v), (c) target or desired concentration of each agent in the composition once delivered in the flow path (e.g., 1 ppm, 2 ppm, 4 ppm, 10 ppm, 50 ppm, 100 ppm, 500 ppm, 1000 ppm or more), (d) the rate of crude oil flow, (e) the rate of injection of the composition, (f) the types of bacteria present in the consortium of the biofilm, (g) the level of bacterial biomass and/or biofilm present, (h) the presence of visible evidence of corrosion (e.g., pits) (which generally is associated with the degree of corrosion in an increasing linear relationship), (i) and the detection of metal loss on test coupons. Each of these factors can be assessed, along with other available factors, to gauge the severity of the biofilm burden and/or the MIC risk and/or the degree of biofilm-associated corrosion. Once the severity of the biofilm biomass or biofilm-associated corrosion is known, the skill person can determine the best course for administering the treatment (i.e., the composition of the present disclosure).

Treatment may be aggressive in nature, or otherwise less aggressive, depending on the degree and severity of the biofilm formation/biomass and/or MIC. For example, if the degree of biofilm-associated corrosion is determined to be low, a gentle treatment may be administered by, for example, reducing the total amount or concentration of indole delivered, reducing the number of hours of continued injection into the site of interest, or increasing the number of days spanning between follow-up injections. However, if the degree of biofilm-associated corrosion is determined to be high, a more aggressive treatment may be administered by, for example, increasing the total amount or concentration of the agents or components of the composition delivered, increasing the time period for continuous injection, or shortening the number of time or days between successive treatments.

The skill person will easily be able to assess the biofilm biomass and/or the degree of biofilm-associated corrosion based on various measurable input and accordingly determine a proper course of treatment using the composition of the disclosed herein without undue experimentation.

### Biofilms and Treatable Surfaces

Without being bound by theory, the present inventors have surprisingly discovery that biofilm formation and maintenance is related to overexpression of prophage gene(s)/protein(s) and promoting prophage protein function, and consequently, modulating prophage gene expression and prophage protein activation (or stability, activation, etc.) may be used to modulate biofilms (e.g., reduce, increase, mitigate, enhance, promote, or eliminate biofilms), and in particular, surfaces (e.g., metal surfaces on equipment or porous surfaces of oil reservoirs) involved in the acquisition, recovery, storage, transport, and refinery in the petrochemical and natural gas industries.

It will be appreciated that microorganisms present in aqueous environments form biofilms on solid surfaces. Biofilm consists of populations of microorganisms and their hydrated polymeric secretions. Numerous types of organisms may exist in any particular biofilm, ranging from strictly aerobic bacteria at the water interface to anaerobic bacteria, such as sulphate reducing bacteria (SRB) at the oxygen depleted metal surface. Biofilm formation is thought to follow a multi-series of specific steps that include: (a) an initial bacterial attachment stage that is rapid and reversible; (b) a longer term attachment stage; (c) a replication phase; (d) a polysaccharide-rich matrix secretion stage; (e) a biofilm maturation stage; and (f) finally bacterial dispersal stage. Biofilms can be microns to millimeters to centimeters or more in thickness and can develop over the course of hours, day, or months, depending on many factors that include the consortium of bacteria present and the environment. Biofilms are highly complex naturally occurring biotic structures having a wide range of characteristics and their exact role in corrosion is still under intense study. However, biofilm-associated corrosion is at least a function of the composition of the underlying bacterial population that forms the biofilm and on the environment. See Kwan Li et al.

The presence of biofilm can contribute to corrosion in at least three ways: (1) physical deposition, (2) production of corrosive by-products, and (3) depolarization of the corrosion cell caused by chemical reaction.

Many of the byproducts of microbial metabolism including organic acids and hydrogen sulphide are corrosive. These materials can concentrate in the biofilm causing accelerated metal attack. Corrosion tends to be self-limited due to the build-up of corrosion reaction products. However, microbes can absorb some of these materials in their metabolism, thereby removing them from the anodic and cathodic sites. The removal of reaction products, termed depolarization, stimulates further corrosion.

Biofilms are usually found on solid substrates submerged in or exposed to an aqueous solution, although they can form as floating mats on liquid surfaces and also on the surface of leaves, particularly in high humidity climates. Given sufficient resources for growth, a biofilm will quickly grow to be macroscopic (visible to the naked eye). Biofilms can contain many different types of microorganism, e.g., bacteria, archaea, protozoa, fungi and algae; each group performs specialized metabolic functions. However, some organisms will form single-species films under certain conditions. The social structure (cooperation, competition) within a biofilm highly depends on the different species present.

Biofilms are held together and protected by a matrix of secreted polymeric compounds called EPS. EPS is an abbreviation for either extracellular polymeric substance or exopolysaccharide, although the latter one only refers to the polysaccharide moiety of EPS. In fact, the EPS matrix consists not only of polysaccharides but also of proteins (which may be the major component in environmental and waste water biofilms) and nucleic acids. A large proportion of the EPS is more or less strongly hydrated, however, hydrophobic EPS also occur; one example is cellulose which is produced by a range of microorganisms. This matrix encases the cells within it and facilitates communication among them through biochemical signals as well as gene exchange. The EPS matrix is an important key to the evolutionary success of biofilms and their resistance to, in this case, biocides and other chemical treatments to remove them. One reason is that it traps extracellular enzymes and keeps them in close proximity to the cells. Thus, the matrix represents an external digestion system and allows for stable synergistic microconsortia of different species (Wingender and Flemming, Nat. Rev. Microbiol. 8, 623-633). Some biofilms have been found to contain water channels that help distribute nutrients and signaling molecules.

Despite these protective physical and biological properties of biofilms—and in particular, the EPS which presents a significant permeability barrier to anti-bacterial agents—the compositions of the present disclosure are believed to be effective in mitigating the formation of biofilms on surfaces (e.g., metal or porous surfaces), in particular, under anaerobic conditions.

The compositions of the disclosure can be used to treat any affected surface, and in particular, any affected metal surface on any equipment involved in the storage, transport, and/or refinery of petroleum and/or natural gas products, or porous surface involved in the acquisition storage, transport, and/or refinery of petroleum and/or natural gas products. For example, affected surfaces can include pipeline that transports crude oil from onshore or offshore drill site or from hydraulic fracturing sites to local or distant petroleum and/or natural gas refineries. Problematic biofilms can form along the interior surfaces of pipelines over distances that extend over many miles or tens of miles, leading to corrosive conditions over a multitude of points. It is generally accepted that pipeline corrosion represents the majority of corrosive damage due to MIC in the oil and gas industries, particularly given that there are over 190,000 miles of liquid pipelines in the US alone. In another example, affected surfaces can include oil storage facilities at refinery sites or those located on oil transport tankers. Other equipment, such as pumps, valves, and other equipment that comes into contact with the oil flow path is susceptible to the formation of biofilms and thus to MIC. Furthermore, the surface to be treated can be a reservoir, in which biofilm modulation is required. For example, the productivity of the reservoir can be enhanced by promoting biofilm production to plug the porous surface of the reservoir. Any and all of these sites and surfaces may be treated using the methods disclosed herein.

### Combination Treatments

The disclosed composition treatment methods are also contemplated to be combined with other MIC-mitigation strategies, such as the use of corrosion-resistant metals, temperature control, pH control, radiation, filtration, protective coatings with corrosion inhibitors or other chemical controls (e.g., biocides, oxidizers, acids, alkalis), bacteriological controls (e.g., phages, enzymes, parasitic bacteria, antibodies, competitive microflora), pigging (i.e., mechanical delamination of corrosion products), anodic and cathodic protection, and modulation of nutrient levels.

In particular, in certain embodiments relating to pipeline treatment, the pipeline is first treated with pigging. The pigging can help not only to physically to remove the biofilm, but also acts to disturb the biofilm such that the permeation of the biofilm is improved, thereby rendering the composition treatment more effective.

Methods and equipment for pigging lines is well known in the art, and can be found described in the following US patents, each of which are incorporated by reference: U.S. Pat. Nos. 9,010,826; 8,858,732; 8,719,989; 7,739,767; 7,275,564; 6,874,757; 6,182,761; and 6,109,829.

### EXAMPLES

This invention is further illustrated by the following examples which should not be construed as limiting. The contents of all references, and published patents and patent applications cited throughout the application are hereby incorporated by reference. Those skilled in the art will recognize that the invention may be practiced with variations on the disclosed structures, materials, compositions and methods, and such variations are regarded as within the ambit of the invention.

### Example 1: Biofilms Overexpress Phage Genes

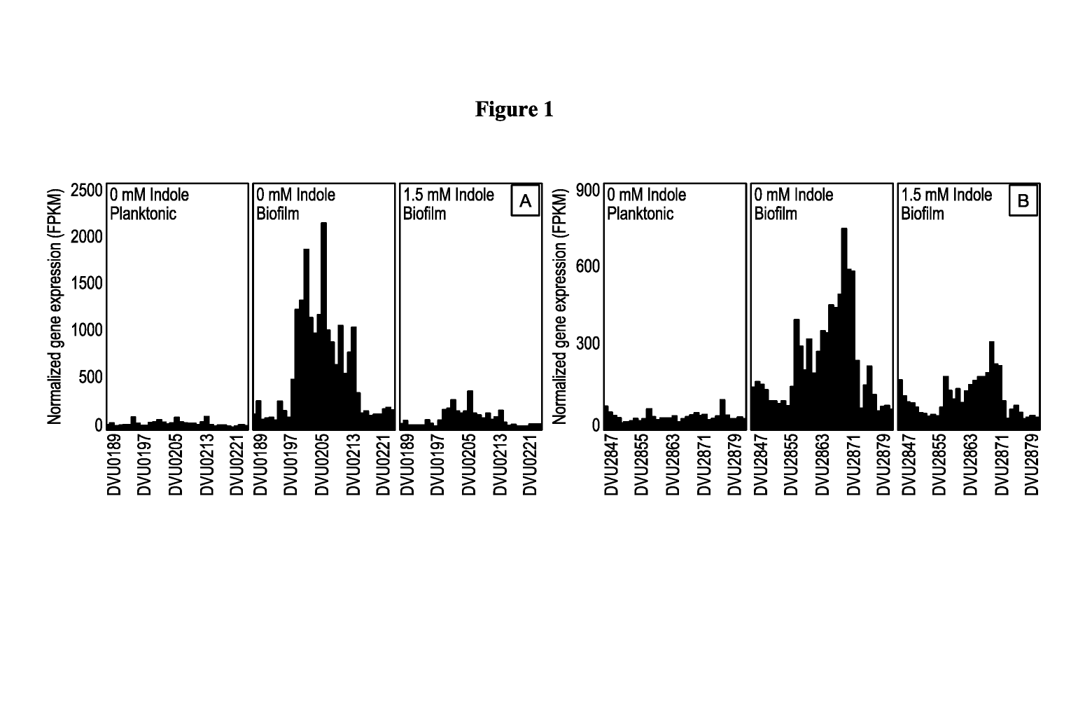

FIG. 1 shows the normalized gene expression (FPKM is fragments per kilobase of transcript per million mapped reads) of two prophage regions (A: DVU0189-DVU0221 and B: DVU2847-DVU2879) in planktonic and biofilm samples. Overexpression of prophages is observed in biofilm samples when compared to planktonic samples. Control of biofilm via indole, as previously demonstrated in U.S. Patent Application Publication 2016-0360749 A1, repressed phage genes expression.

### Example 2. Occurrence of Prophages in Different Bacteria

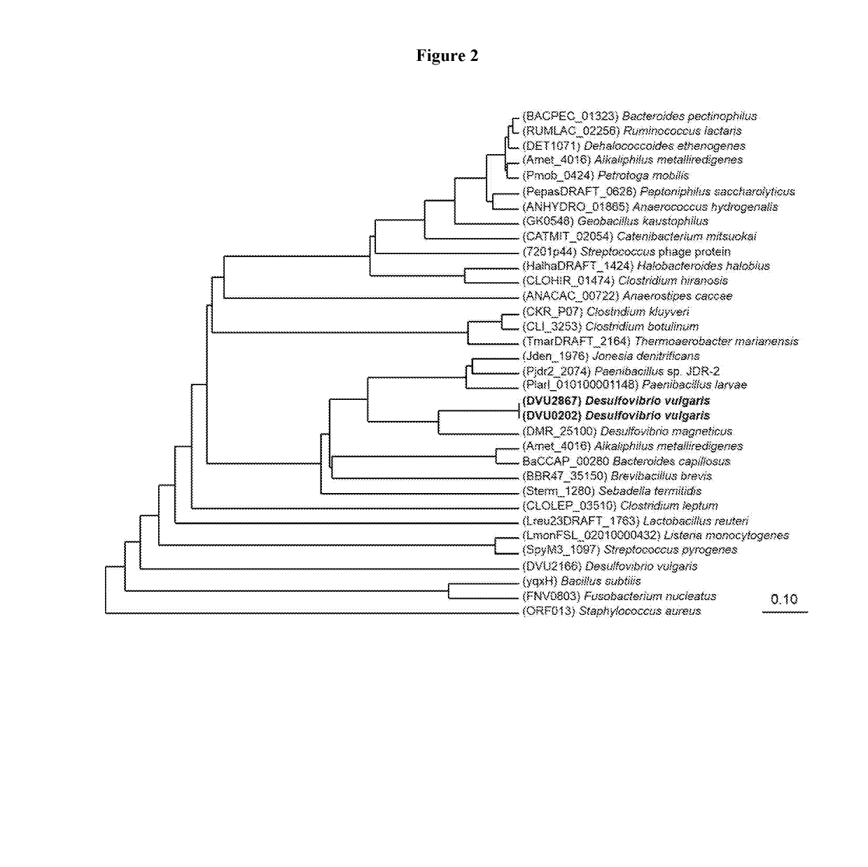

FIG. 2 shows the phylogenetic affiliation of two prophage regions selected from Desulfovibrio vulgaris in Example 1 (DVU0202 and DVU2867, in bold) with 33 exemplary bacterial species. The prophage nomenclature is shown in parenthesis with the microbial host genus and species in italic. The maximum likelihood tree from protein sequences was calculated using PHYML as implemented in ARB (W. Ludwig et al., Nucleic Acids Research, 2004; 32(4): 1363-1371). The scale bar indicates 10% sequences divergence.

### Specific Embodiments

In an aspect, the disclosure provides a method of detecting biofilm formation or monitoring the state of a biofilm. The method comprising: providing a test sample; detecting at least one of a prophage gene, a prophage gene expression, a prophage gene transcript, a prophage protein, a prophage metabolite, a prophage intermediate, or a combination thereof, wherein the existence, amount or level of prophage gene expression, the prophage gene transcript, the prophage protein, the prophage metabolite, the prophage intermediate, or the combination thereof relative to a control is indicative of biofilm presence, formation or the state of the biofilm in a sample.

In any of the aspects or embodiments described herein, an increase relative to levels detected in a previously taken sample is indicative of biofilm formation or an increase in biofilm burden/biomass.

In any of the aspects or embodiments described herein, a decrease relative to levels detected in a previously taken sample is indicative of a decrease in biofilm burden/biomass.

In any of the aspects or embodiments described herein, an increase relative to levels observed in planktonic bacteria is indicative of biofilm formation.

In any of the aspects or embodiments described herein, a lack of detection is indicative of a lack of a biofilm.

In any of the aspects or embodiments described herein, the step of detecting comprises binding at least one of: (i) at least one labelled nucleic acid probe that hybridizes to the prophage gene, the prophage transcript, or a combination thereof (ii) at least one binding polypeptide that binds the prophage protein, the prophage metabolite, the prophage intermediate, or a combination thereof or (iii) a combination thereof.

In any of the aspects or embodiments described herein, (ii) can further comprise: contacting the sample with the binding polypeptide; and contacting the sample with a labelled secondary polypeptide that binds specifically to the binding polypeptide.

In any of the aspects or embodiments described herein, the binding polypeptide includes an antibody or an antigen-binding fragment thereof.

In any of the aspects or embodiments described herein, the label is a fluorophore, an enzyme, a radioisotope, or a chemiluminescent compound.

In any of the aspects or embodiments described herein, the biofilm is formed by an anaerobic bacteria.

In any of the aspects or embodiments described herein, the anaerobic bacteria is selected from the group consisting of sulfate reducing bacteria, iron oxidizing bacteria, sulfur oxidizing bacteria, nitrate reducing bacteria, methanogens, and acid producing bacteria.

In any of the aspects or embodiments described herein, the sulfate reducing bacteria is of the genera Desulfovibrio, Desulfotomaculum, Desulfosporomusa, Desulfosporosinus, Desulfobacter, Desulfobacterium, Desulfobacula, Desulfobotulus, Desulfocella, Desulfococcus, Desulfofaba, Desulfofrigus, Desulfonema, Desulfosarcina, Desulfospira, Desulfotalea, Desulfotignum, Desulfobulbus, Desulfocapsa, Desulfofustis, Desulforhopalis, Desulfoarculus, Desulfobacca, Desulfomonile, Desulfotigmum, Desulfohalobium, Desulfomonas, Desulfonatronovibrio, Desulfomicrobium, Desulfonatronum, Desulfacinum, Desulforhabdus, Syntrophobacter, Syntrophothermus, Thermaerobacter, and Thermodesulforhabdus.

In any of the aspects or embodiments described herein, the sulfate reducing bacteria is of the genera Desulfovibrio.

In a further aspect, the description provides a method of modulating bacteria. The method comprises contacting the bacteria with an effective amount of a composition comprising at least one agent that: (a) activates or promotes at least one of a prophage gene, prophage gene transcription, a prophage gene expression, a prophage transcript, a prophage protein, a prophage metabolite, a prophage intermediate or a combination thereof; or (b) inhibits or inactivates at least one of a prophage gene, prophage gene transcription, a prophage gene expression, a prophage transcript, a prophage protein, a prophage metabolite, a prophage intermediate or a combination thereof; or (c) activates or promotes at least one of a prophage repressor gene, prophage repressor gene transcription, a prophage repressor transcript, a prophage repressor protein or a combination thereof; (d) inhibits or inactivates at least one of a prophage repressor gene, prophage repressor gene transcription, a prophage repressor transcript, a prophage repressor protein or a combination thereof; or (e) a combination thereof.

In any of the aspects or embodiments described herein, the method further comprises detecting biofilm formation or monitoring the state of a biofilm in accordance with the method described herein.

In any of the aspects or embodiments described herein, (a) activating or promoting of the prophage gene, prophage gene transcription, the prophage gene expression, the prophage transcript, the prophage protein, the prophage metabolite, the prophage intermediate or a combination thereof results in the activation or induction of a prophage lytic cycle or inhibition of a prophage lysogenic cycle in the bacteria; or (b) inhibiting or inactivating a prophage gene, prophage gene transcription, a prophage gene expression, a prophage transcript, a prophage protein, a prophage metabolite, a prophage intermediate or a combination thereof results in the inhibition of a prophage lytic cycle or promotion of a prophage lysogenic phase in the bacteria; or (c) activating or promoting a prophage repressor gene, prophage repressor gene transcription, a prophage repressor gene transcript, a prophage repressor protein or a combination thereof results in the inhibition of a prophage lytic cycle or promotion of a prophage lysogenic phase in the bacteria; or (d) inhibiting or inactivating a prophage repressor gene, prophage repressor gene transcription, a prophage repressor gene transcript, a prophage repressor protein or a combination thereof results in the activation or induction of a prophage lytic cycle or inhibition of a prophage lysogenic cycle in the bacteria.

In any of the aspects or embodiments described herein, the activation or induction of the prophage lytic cycle or the inhibition a prophage lysogenic cycle results in a reduction in biofilm formation or burden.

In any of the aspects or embodiments described herein, inhibiting of the prophage lytic cycle or promoting the prophage lysogenic phase results in an increase in biofilm formation or burden.

In any of the aspects or embodiments described herein, the agent or agents induce the prophage lytic cycle or inhibit biofilm production/maintenance or promotes biofilm degradation.

In any of the aspects or embodiments described herein, the method further comprises applying a secondary treatment for reducing the formation or activity of a biofilm on a surface.

In any of the aspects or embodiments described herein, the secondary treatment is selected from the group consisting of pigging, radiation treatment, pH adjustment, nutrient adjustment, and installation of corrosion-resistant metals.

In yet another aspect, the disclosure provides a composition that comprises an effective amount of an agent which: (i) inhibits the transcription of at least one prophage gene or promotes the transcription of at least one prophage repressor gene; or (ii) inhibits the translation of at least one prophage gene transcript or promotes the translation of at least one prophage repressor gene transcript; or (iii) leads to the degradation of at least one prophage protein or inhibits the activity of at least one prophage protein or stabilizes of at least one prophage repressor protein or enhances the activity of at least one prophage repressor protein; or (iv) leads to the degradation of at least one prophage metabolite or inhibits the activity of at least one prophage metabolite or stabilizes of at least one prophage repressor metabolite or enhances the activity of at least one prophage repressor metabolite; or (v) leads to the degradation of at least one intermediate or inhibits the activity of at least one prophage intermediate or stabilizes of at least one prophage repressor intermediate or enhances the activity of at least one prophage repressor intermediate.

In any of the aspects or embodiments described herein, the agent is an antibody or a binding fragment thereof.

In any of the aspects or embodiments described herein, the agent is a nucleic acid that binds to at least one of the prophage gene, the prophage transcript or a combination thereof.

In any of the aspects or embodiments described herein, the composition further comprises at least one additional agent.

In any of the aspects or embodiments described herein, the additional agent is an indole or a functionally equivalent analog or derivative thereof in an aqueous composition or a non-aqueous composition.

In any of the aspects or embodiments described herein, the additional agent is an indole or a functionally equivalent analog or derivative thereof in a liquid composition with an acidic pH or a basic pH.

In any of the aspects or embodiments described herein, the additional agent is a biocide selected from the group consisting of germicides, antibiotics, antibacterials, antivirals, antifungals, antiprotozoals and antiparasites.

In any of the aspects or embodiments described herein, the effective amount of the composition comprising indole or a functionally equivalent analog or derivative thereof provides a concentration of indole that is between about 50-500 micromolar, about 0.5-1.0 mM, about 1.0 mM-5 mM, about 2.5 mM-10 mM, about 5 mM-25 mM, about 10 mM-100 mM, or about 50 mM-1000 mM.

In an additional aspect, the disclosure provides a method for mitigating or eliminating Microbial Influenced Corrosion of a metal surface. The method comprises contacting the metal surface with an effective amount of a liquid composition comprising at least one agent that: (i) inhibits at least one of a prophage gene expression, a prophage transcript, a prophage protein, a prophage metabolite, a prophage intermediate, or a combination thereof; (ii) inactivates a prophage gene; or (iii) a combination thereof.

In any of the aspects or embodiments described herein, the Microbial Influenced Corrosion is caused by a bacterial biofilm deposited on the surface of the metal surface.

### PCT/EP Clauses

1. A method of detecting biofilm formation or monitoring the state of a biofilm, the method comprising:

providing a test sample;

detecting at least one of a prophage gene, a prophage gene expression, a prophage gene transcript, a prophage protein, a prophage metabolite, a prophage intermediate, or a combination thereof,

wherein the existence, amount or level of prophage gene expression, the prophage gene transcript, the prophage protein, the prophage metabolite, the prophage intermediate, or the combination thereof relative to a control is indicative of biofilm presence, formation or the state of the biofilm in a sample.

2. The method of clause 1, wherein:

an increase relative to levels detected in a previously taken sample is indicative of biofilm formation or an increase in biofilm burden/biomass;

a decrease relative to levels detected in a previously taken sample is indicative of a decrease in biofilm burden/biomass;

an increase relative to levels observed in planktonic bacteria is indicative of biofilm formation; or a lack of detection is indicative of a lack of a biofilm.

3. The method of clause 1 or 2, wherein the step of detecting comprises binding at least one of:

(i) at least one labelled nucleic acid probe that hybridizes to the prophage gene, the prophage transcript, or a combination thereof;

(ii) at least one binding polypeptide that binds the prophage protein, the prophage metabolite, the prophage intermediate, or a combination thereof by (a) contacting the sample with the binding polypeptide and contacting the sample with a labelled secondary polypeptide that binds specifically to the binding polypeptide; or

(iii) a combination thereof.

4. The method of clause 3, wherein the binding polypeptide includes an antibody or an antigen-binding fragment thereof.

5. The method of clause 3, wherein the label is a fluorophore, an enzyme, a radioisotope, or a chemiluminescent compound.

6. The method of clause 3, wherein the biofilm is formed by an anaerobic bacteria.

7. A method of modulating bacteria, the method comprising contacting the bacteria with an effective amount of a composition comprising at least one agent that:

(a) activates or promotes at least one of a prophage gene, prophage gene transcription, a prophage gene expression, a prophage transcript, a prophage protein, a prophage metabolite, a prophage intermediate or a combination thereof; or

(b) inhibits or inactivates at least one of a prophage gene, prophage gene transcription, a prophage gene expression, a prophage transcript, a prophage protein, a prophage metabolite, a prophage intermediate or a combination thereof; or

(c) activates or promotes at least one of a prophage repressor gene, prophage repressor gene transcription, a prophage repressor transcript, a prophage repressor protein or a combination thereof;

(d) inhibits or inactivates at least one of a prophage repressor gene, prophage repressor gene transcription, a prophage repressor transcript, a prophage repressor protein or a combination thereof; or

(e) a combination thereof.

8. The method of clause 7, further comprising detecting biofilm formation or monitoring the state of a biofilm in accordance with any of clauses 1-6.

9. The method of clause 7 or 8, wherein:

(a) activating or promoting of the prophage gene, prophage gene transcription, the prophage gene expression, the prophage transcript, the prophage protein, the prophage metabolite, the prophage intermediate or a combination thereof results in the activation or induction of a prophage lytic cycle or inhibition of a prophage lysogenic cycle in the bacteria; or

(b) inhibiting or inactivating a prophage gene, prophage gene transcription, a prophage gene expression, a prophage transcript, a prophage protein, a prophage metabolite, a prophage intermediate or a combination thereof results in the inhibition of a prophage lytic cycle or promotion of a prophage lysogenic phase in the bacteria; or

(c) activating or promoting a prophage repressor gene, prophage repressor gene transcription, a prophage repressor gene transcript, a prophage repressor protein or a combination thereof results in the inhibition of a prophage lytic cycle or promotion of a prophage lysogenic phase in the bacteria; or

(d) inhibiting or inactivating a prophage repressor gene, prophage repressor gene transcription, a prophage repressor gene transcript, a prophage repressor protein or a combination thereof results in the activation or induction of a prophage lytic cycle or inhibition of a prophage lysogenic cycle in the bacteria.

10. The method of clause 9, wherein:

the activation or induction of the prophage lytic cycle or the inhibition a prophage lysogenic cycle results in a reduction in biofilm formation or burden; or

inhibiting of the prophage lytic cycle or promoting the prophage lysogenic phase results in an increase in biofilm formation or burden.

11. The method of any of clauses 7-19, wherein the agent or agents induce the prophage lytic cycle or inhibit biofilm production/maintenance or promotes biofilm degradation.

12. The method of any of clauses 7-11, further comprising applying a secondary treatment for reducing the formation or activity of a biofilm on a surface, the secondary treatment is selected from the group consisting of pigging, radiation treatment, pH adjustment, nutrient adjustment, and installation of corrosion-resistant metals.

13. A composition comprising an effective amount of an agent which:

(i) inhibits the transcription of at least one prophage gene or promotes the transcription of at least one prophage repressor gene; or

(ii) inhibits the translation of at least one prophage gene transcript or promotes the translation of at least one prophage repressor gene transcript; or

(iii) leads to the degradation of at least one prophage protein or inhibits the activity of at least one prophage protein or stabilizes of at least one prophage repressor protein or enhances the activity of at least one prophage repressor protein; or

(iv) leads to the degradation of at least one prophage metabolite or inhibits the activity of at least one prophage metabolite or stabilizes of at least one prophage repressor metabolite or enhances the activity of at least one prophage repressor metabolite; or

(v) leads to the degradation of at least one intermediate or inhibits the activity of at least one prophage intermediate or stabilizes of at least one prophage repressor intermediate or enhances the activity of at least one prophage repressor intermediate.

14. The composition of clause 13, wherein the agent is at least one of:

an antibody or a binding fragment thereof;

a nucleic acid that binds to the prophage gene;

a nucleic acid that binds to the prophage transcript;

or a combination thereof.

15. The composition of clause 13 or 14, wherein the composition further comprises at least one additional agent selected from the group consisting of germicides, antibiotics, antibacterials, antivirals, indole or a functionally equivalent analog or derivative thereof, antifungals, antiprotozoals and antiparasites.

16. A method for mitigating or eliminating Microbial Influenced Corrosion of a metal surface comprising contacting the metal surface with an effective amount of a liquid composition comprising at least one agent that:

(i) inhibits at least one of a prophage gene expression, a prophage transcript, a prophage protein, a prophage metabolite, a prophage intermediate, or a combination thereof;

(ii) inactivates a prophage gene; or

(iii) a combination thereof,

wherein the Microbial Influenced Corrosion is caused by a bacterial biofilm deposited on the surface of the metal surface.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

1. A method of detecting biofilm formation or monitoring the state of a biofilm, the method comprising:
providing a test sample;
detecting at least one of a prophage gene, a prophage gene expression, a prophage gene transcript, a prophage protein, a prophage metabolite, a prophage intermediate, or a combination thereof,
wherein the existence, amount or level of prophage gene expression, the prophage gene transcript, the prophage protein, the prophage metabolite, the prophage intermediate, or the combination thereof relative to a control is indicative of biofilm presence, formation or the state of the biofilm in a sample.

2. The method of claim 1, wherein an increase relative to levels detected in a previously taken sample is indicative of biofilm formation or an increase in biofilm burden/biomass.

3. The method of claim 1, wherein a decrease relative to levels detected in a previously taken sample is indicative of a decrease in biofilm burden/biomass.

4. The method of claim 1, wherein an increase relative to levels observed in planktonic bacteria is indicative of biofilm formation.

5. The method of claim 1, wherein a lack of detection is indicative of a lack of a biofilm.

6. The method of claim 1, wherein the step of detecting comprises binding at least one of:
(i) at least one labelled nucleic acid probe that hybridizes to the prophage gene, the prophage transcript, or a combination thereof;
(ii) at least one binding polypeptide that binds the prophage protein, the prophage metabolite, the prophage intermediate, or a combination thereof; or
(iii) a combination thereof.

7. The method of claim 6, wherein (ii) further comprises:
contacting the sample with the binding polypeptide; and
contacting the sample with a labelled secondary polypeptide that binds specifically to the binding polypeptide.

8. The method of claim 7, wherein the binding polypeptide includes an antibody or an antigen-binding fragment thereof.

9. The method of claim 6, wherein the label is a fluorophore, an enzyme, a radioisotope, or a chemiluminescent compound.

10. The method of claim 1, wherein the biofilm is formed by an anaerobic bacteria.

11. The method of claim 10, wherein the anaerobic bacteria is selected from the group consisting of sulfate reducing bacteria, iron oxidizing bacteria, sulfur oxidizing bacteria, nitrate reducing bacteria, methanogens, and acid producing bacteria.

12. The method of claim 11, wherein the sulfate reducing bacteria is of the genera Desulfovibrio, Desulfotomaculum, Desulfosporomusa, Desulfosporosinus, Desulfobacter, Desulfobacterium, Desulfobacula, Desulfobotulus, Desulfocella, Desulfococcus, Desulfofaba, Desulfofrigus, Desulfonema, Desulfosarcina, Desulfospira, Desulfotalea, Desulfotignum, Desulfobulbus, Desulfocapsa, Desulfofustis, Desulforhopalis, Desulfoarculus, Desulfobacca, Desulfomonile, Desulfotigmum, Desulfohalobium, Desulfomonas, Desulfonatronovibrio, Desulfomicrobium, Desulfonatronum, Desulfacinum, Desulforhabdus, Syntrophobacter, Syntrophothermus, Thermaerobacter, and Thermodesulforhabdus.

13. The method claim of claim 12, wherein the sulfate reducing bacteria is of the genera Desulfovibrio.

14. A method of modulating bacteria, the method comprising contacting the bacteria with an effective amount of a composition comprising at least one agent that:
(a) activates or promotes at least one of a prophage gene, prophage gene transcription, a prophage gene expression, a prophage transcript, a prophage protein, a prophage metabolite, a prophage intermediate or a combination thereof; or
(b) inhibits or inactivates at least one of a prophage gene, prophage gene transcription, a prophage gene expression, a prophage transcript, a prophage protein, a prophage metabolite, a prophage intermediate or a combination thereof; or
(c) activates or promotes at least one of a prophage repressor gene, prophage repressor gene transcription, a prophage repressor transcript, a prophage repressor protein or a combination thereof; or
(d) inhibits or inactivates at least one of a prophage repressor gene, prophage repressor gene transcription, a prophage repressor transcript, a prophage repressor protein or a combination thereof; or
(e) a combination thereof.

15. The method of claim 14, further comprising detecting biofilm formation or monitoring the state of a biofilm in accordance with claim 1.

16. The method of claim 14, wherein:
(a) activating or promoting of the prophage gene, prophage gene transcription, the prophage gene expression, the prophage transcript, the prophage protein, the prophage metabolite, the prophage intermediate or a combination thereof results in the activation or induction of a prophage lytic cycle or inhibition of a prophage lysogenic cycle in the bacteria; or
(b) inhibiting or inactivating a prophage gene, prophage gene transcription, a prophage gene expression, a prophage transcript, a prophage protein, a prophage metabolite, a prophage intermediate or a combination thereof results in the inhibition of a prophage lytic cycle or promotion of a prophage lysogenic phase in the bacteria; or
(c) activating or promoting a prophage repressor gene, prophage repressor gene transcription, a prophage repressor gene transcript, a prophage repressor protein or a combination thereof results in the inhibition of a prophage lytic cycle or promotion of a prophage lysogenic phase in the bacteria; or
(d) inhibiting or inactivating a prophage repressor gene, prophage repressor gene transcription, a prophage repressor gene transcript, a prophage repressor protein or a combination thereof results in the activation or induction of a prophage lytic cycle or inhibition of a prophage lysogenic cycle in the bacteria.

17. The method of claim 16, wherein the activation or induction of the prophage lytic cycle or the inhibition a prophage lysogenic cycle results in a reduction in biofilm formation or burden.

18. The method of claim 16, wherein inhibiting of the prophage lytic cycle or promoting the prophage lysogenic phase results in an increase in biofilm formation or burden.

19. The method of claim 14, wherein the agent or agents induce the prophage lytic cycle or inhibit biofilm production/maintenance or promotes biofilm degradation.

20. The method of claim 14, further comprising applying a secondary treatment for reducing the formation or activity of a biofilm on a surface.

21. The method of claim 20, wherein the secondary treatment is selected from the group consisting of pigging, radiation treatment, pH adjustment, nutrient adjustment, and installation of corrosion-resistant metals.

22. A composition comprising an effective amount of an agent which:
(i) inhibits the transcription of at least one prophage gene or promotes the transcription of at least one prophage repressor gene; or
(ii) inhibits the translation of at least one prophage gene transcript or promotes the translation of at least one prophage repressor gene transcript; or
(iii) leads to the degradation of at least one prophage protein or inhibits the activity of at least one prophage protein or stabilizes of at least one prophage repressor protein or enhances the activity of at least one prophage repressor protein; or
(iv) leads to the degradation of at least one prophage metabolite or inhibits the activity of at least one prophage metabolite or stabilizes of at least one prophage repressor metabolite or enhances the activity of at least one prophage repressor metabolite; or
(v) leads to the degradation of at least one intermediate or inhibits the activity of at least one prophage intermediate or stabilizes of at least one prophage repressor intermediate or enhances the activity of at least one prophage repressor intermediate.

23. The composition of claim 22, wherein the agent is an antibody or a binding fragment thereof.

24. The composition of claim 22, wherein the agent is a nucleic acid that binds to at least one of the prophage gene, the prophage transcript or a combination thereof.

25. The composition of claim 22, wherein the composition further comprises at least one additional agent.

26. The composition of claim 25, wherein the additional agent is an indole or a functionally equivalent analog or derivative thereof in an aqueous composition or a non-aqueous composition.

27. The composition of claim 25, wherein the additional agent is an indole or a functionally equivalent analog or derivative thereof in a liquid composition with an acidic pH or a basic pH.

28. The composition of claim 25, wherein the additional agent is a biocide selected from the group consisting of germicides, antibiotics, antibacterials, antivirals, antifungals, antiprotozoals and antiparasites.

29. The composition of claim 26, wherein the effective amount of the composition comprising indole or a functionally equivalent analog or derivative thereof provides a concentration of indole that is between 50-500 micromolar.

30. A method for mitigating or eliminating Microbial Influenced Corrosion of a metal surface comprising contacting the metal surface with an effective amount of a liquid composition comprising at least one agent that:
(i) inhibits at least one of a prophage gene expression, a prophage transcript, a prophage protein, a prophage metabolite, a prophage intermediate, or a combination thereof;
(ii) inactivates a prophage gene; or
(iii) a combination thereof.

31. The method of claim 30, wherein the Microbial Influenced Corrosion is caused by a bacterial biofilm deposited on the surface of the metal surface.
